(19) United States
(12) Patent Application Publication
(10) Pub. No.: US 2015/0039240 A1
(43) Pub. Date: Feb. 5, 2015

(54) PROTEIN IDENTIFICATION METHOD AND SYSTEM

(71) Applicant: SHIMADZU CORPORATION, Kyoto-shi (JP)

(72) Inventors: Akiyasu YOSHIZAWA, Kyoto-shi (JP); Hiroki KUYAMA, Kyoto-shi (JP)

(73) Assignee: SHIMADZU CORPORATION, Kyoto-shi (JP)

(21) Appl. No.: 14/450,687

(22) Filed: Aug. 4, 2014

(30) Foreign Application Priority Data
Aug. 5, 2013 (JP) 2013-162008

(51) Int. Cl. G06F 19/18; G01N 33/68; G06F 19/28

(52) U.S. Cl. CPC G06F 19/18; G06F 19/28; G01N 33/6827
USPC 702/19

(57) ABSTRACT

When a terminal sequence to be investigated is specified (S1), a corresponding protein is extracted from a database containing known proteins linked with terminal sequences (S2). If one protein cannot be uniquely identified (“No” in S5), amino-acid residues to be bonded to the target terminal sequences are selected, and new terminal sequences with the selected amino-acid residues respectively added are created for further investigation (S7). Each new terminal sequence is examined as to whether or not one protein can be uniquely identified from it, and if not so, another amino-acid residue is added. While such processes are repeated, if it has been found that the protein can be uniquely identified before the sequence length reaches an upper limit, the sequence length is displayed (S9). If the sequence length has reached the upper limit without successful identification, a message saying that the protein cannot be identified is displayed (S11).

### TECHNICAL FIELD

The present invention relates to a method for identifying a protein to be analyzed by deducing an amino-acid sequence of a peptide using mass spectrometric data obtained by a mass spectrometry of a test sample containing a mixture of peptides originating from the protein to be analyzed. It also relates to a system for such a method.

### BACKGROUND ART

In recent years, the technique of proteomic analysis for comprehensively analyzing proteins has been widely used. It has been making dramatic technical advancements. In the field of proteomic analysis, a commonly known technique for identifying proteins using a matrix-assisted laser desorption/ionization time-of-flight mass spectrometer (MALDI-TOFMS) or other types of mass spectrometers is the database search. A general procedure of the database search is as follows: A peak list containing mass-to-charge ratio information (and, in some cases, intensity information) of each peak located in an MSⁿ spectrum obtained by an MSⁿ analysis of a sample containing a mixture of peptides originating from a protein (where n is an integer equal to or greater than two) is created. This peak list is compared with reference information, such as the mass-to-charge ratios theoretically calculated from proteins registered in a database or a peak list of proteins obtained by actual measurements. Using the degree of matching of the peaks as a clue, the amino-acid sequence of each peptide is determined, and based on the determination result, the original protein is identified.

In many cases, proteins have their terminal regions cut or modified. Therefore, in a study of a protein, it is extremely important to correctly determine the amino-acid sequences of its terminal regions. However, with the previously described common techniques, it is often the case that the amino-acid sequences of the terminal regions (especially, the C-terminal) of a protein cannot be easily detected and their analysis is difficult to perform. To address this problem, various techniques for an amino-acid sequence analysis of the terminal regions of proteins have been conventionally developed or proposed.

For example, Non Patent Literature 1 discloses a technique in which a fragment of an amino-acid sequence of an N-terminal peptide is identified by a commonly known peptide mass fingerprinting (PMF) method or an MS/MS ion search using Mascot (the database search engine provided by Matrix Science) or a similar program. However, this approach has the problem that proteins can be identified only when the terminal sequence in question is contained in the database being used; if the identification has resulted in failure, no useful information other than the fact that the protein cannot be identified is obtainable by the analysis operator.

Non Patent Literature 2 and Patent Literature 1 disclose a technique in which the entire amino-acid sequence of a protein is determined from a terminal sequence using the de novo sequencing so that the protein can be identified even if the terminal amino-acid sequence being searched for is not contained in the database.

Non Patent Literature 3 is the document that is referenced in all of the previously cited literatures. In this literature, five organisms (Saccharomyces cerevisiae, Escherichia coli, Bacillus subtilis, Mycoplasma genitalium, and human) have been studied regarding the lengths of the N-terminal and C-terminal sequences as well as the specificity (i.e. uniqueness) of the amino-acid sequences. A comparatively affirmative hypothesis has been derived as to whether or not the proteins can be uniquely identified from the respective amino-acid sequences. That is to say, the knowledge obtained in Non Patent Literature 3 based on the experimental result is that “a protein can be uniformly identified by determining the sequences of the terminals of the protein up to a certain length.” It can be said that the techniques described in Non Patent Literature 2 and Patent-Literature 1 are also based on this knowledge.

However, in view of the speed of the technical advancement in this field, Non Patent Literature 3 is a considerably old reference of literature; at the time of the research, only one organism (Mycoplasma) out of the five organisms studied had its genome completely sequenced and published. Furthermore, for the four other organisms, the number of proteins studied was significantly smaller than that of genes (i.e. proteins) constituting the entire genome. For those kinds of organisms whose genome have not been entirely investigated, it is not certain that the derived conclusion is highly reliable.

The present inventors have conducted similar research on the human gene sequences, which revealed that 90% or more of the genes in the entire human genome can be uniquely identified only by the terminal sequences of the proteins derived from the corresponding genes. Accordingly, it can be said that the technique of identifying proteins based on the knowledge that “a protein can be uniformly identified by determining the sequences of the terminals of the protein up to a certain length” is generally a feasible methodology.

However, the research also revealed that a few to several percent of the genes constituting the entire human genome have the same terminal sequences over a length of 20 or more amino-acid residues. For such genes, it is impossible to uniquely identify proteins from only their terminal sequences even if the candidates are narrowed to such proteins whose molecular weights can be measured with a mass spectrometer. Thus, there are a certain number of cases (though not so many) in which the protein identification based on the previously described conventional empirical knowledge cannot be applied.

Such cases are not taken into account in the protein identification methods described in Non Patent Literature 2 or Patent Literature 1; when a protein has not been uniquely identified from the terminal sequence obtained by the mass spectrometry, the analysis operator is not provided with any information as to whether the protein may possibly be identified if the terminal sequence consisting of a slightly larger number of amino acids is known or the protein will not be identified even if the terminal sequence is determined up to the upper-limit length that can be determined through a measurement using the mass spectrometer.

### CITATION LIST

### Patent Literature

Patent Literature 1: JP 2009-132649 A

### Non Patent Literature

Non Patent Literature 1: L. McDonald et al., “Positional proteomics: selective recovery and analysis of N-terminal proteolytic peptides”, Nature Methods, December 2005, Vol. 2, No. 12, pp. 955-957

Non Patent Literature 2: H. Kuyama et al., “A simple and highly successful C-terminal sequence analysis of proteins by mass spectrometry”, Proteomics, 2008, Vol. 8, pp. 1539-1550

Non Patent Literature 3: Marc R. Wilkins et al., “Protein Identification with N and C-Terminal Sequence Tags in Proteome Project”, Journal of Molecular Biology, 1998, Vol. 278, pp. 599-608

### SUMMARY OF INVENTION

### Technical Problem

As already described, in any of the conventional methods for identifying a protein by a terminal sequence revealed by performing a mass spectrometry and analyzing its result, if the protein has not been successfully identified, the obtained result merely indicates there are a plurality of sequences having the given terminal sequence and the protein cannot be identified if no additional effort is made. The result does not provide any additional information, for example, even such basic information that tells whether or not the target being analyzed can be identified only by a terminal sequence. Therefore, there is the possibility that a protein which is actually unidentifiable by a terminal sequence is mistakenly recognized as identifiable and is eventually identified only by the terminal sequence. Conversely, it is also possible that, for a protein which is actually identifiable by a terminal sequence, an additional attempt is made to investigate other amino-acid sequences, which unnecessarily decreases the working efficiency.

The present invention has been developed to solve such a problem, and its objective is to provide a protein identification method and system which can provide correct information as to whether or not a protein which has not been uniquely identified by a given terminal sequence may possibly be identified if a longer terminal sequence of the protein is determined.

### Solution to Problem

The protein identification method according to the present invention aimed at solving the previously described problem is a method for identifying a protein by a terminal sequence which is an amino-acid sequence of a terminal region of the protein, the method including:
a) an identification candidate extraction step, in which a protein corresponding to a target terminal sequence given as a target to be investigated is searched for and extracted as a protein candidate, using information in which various known kinds of proteins are linked with, at least, their respective terminal sequences;
b) an identification result determination step, in which, when only one protein candidate is extracted in the identification candidate extraction step, the candidate is selected as a protein identification result corresponding to the target terminal sequence; and
c) an identification possibility prediction step, in which, when a plurality of protein candidates are extracted in the identification candidate extraction step, an extended length of the terminal sequence with which the protein can be uniquely identified is predicted by determining an amino-acid residue that can be further added to the target terminal sequence with reference to the terminal sequences of the plurality of protein candidates, adding the amino-acid residue to the target terminal sequence to create an extended terminal sequence, and determining, for each extended terminal sequence thus created, whether or not the proteins having the extended terminal sequence can be refined to a single candidate.

The protein identification system according to the present invention aimed at solving the previously described problem, which embodies the previously described protein identification method according to the present invention, is a system for identifying a protein by a terminal sequence which is an amino-acid sequence of a terminal region of the protein, the system including:
a) an identification candidate extractor for searching a database for a protein corresponding to a target terminal sequence given as a target to be investigated using information in which various known kinds of proteins are linked with, at least, their respective terminal sequences, and for extracting the protein as a protein candidate;
b) an identification result determiner for, when only one protein candidate is extracted by the identification candidate extractor, determining the protein candidate as a protein identification result corresponding to the target terminal sequence; and
c) an identification possibility predictor for, when a plurality of protein candidates are extracted by the identification candidate extractor, predicting an extended length of the terminal sequence with which the protein can be uniquely identified by determining an amino-acid residue that can be further added to the target terminal sequence with reference to the terminal sequences of the plurality of protein candidates, adding the amino-acid residue to the target terminal sequence to create an extended terminal sequence, and determining, for each extended terminal sequence thus created, whether or not the proteins having the extended terminal sequence can be refined to a single candidate.

In the protein identification method and system according to the present invention, the “target terminal sequence given as a target to be investigated” may be one of the amino-acid sequences of peptides or peptide fragments deduced from a mass spectrum (including an MSⁿ spectrum, where n is equal to or greater than two) acquired as a result of mass spectrometry of a mixture of peptides obtained by breaking a protein to be identified using, for example, a digestive enzyme. To deduce an amino-acid sequence of a peptide or peptide fragment from a mass spectrum, commonly known methods can be used, such as the peptide mass fingerprinting, or peptide fragment fingerprinting (MS/MS ion search).

Specifically, for example, an analysis operator may select an amino-acid sequence which is likely to be a terminal sequence, and specify or enter that sequence as the target terminal sequence. Alternatively, the selection may be automatically made, without a manual operation by an analysis operator, in such a manner that the terminal sequences of a plurality of (normally, a large number of) protein candidates extracted by a database search or similar process based on a measured mass spectrum are searched for an amino-acid sequence which matches the amino-acid sequence of a peptide or peptide fragment derived from the measured mass spectrum, and the thereby found amino-acid sequence is selected and set as the target terminal sequence. The target terminal sequence does not always need to be derived from a result of a mass spectrometry of a test sample; for example, it may be a terminal sequence entered by an analysis operator who investigates proteins having that amino-acid sequence in its terminal region.

In the system for carrying out the protein identification method according to the present invention, after a target terminal sequence to be investigated is set, the identification candidate extractor searches a database for a protein having the target terminal sequence in its terminal region using information in which various known kinds of proteins are linked, at least, with their respective terminal sequences. By this process, all proteins that satisfy the condition are extracted as protein candidates. If there is only one protein candidate extracted, this candidate is most likely to be the correct protein. In such a case, the identification result determiner adopts that single candidate as the protein identification result corresponding to the target terminal sequence. The result is presented, for example, on a display unit.

If there are a plurality of protein candidates extracted, it is impossible to uniquely identify the protein by the currently set target terminal sequence. In such a case, the identification possibility predictor estimates how many amino-acid residues must be revealed in addition to the currently set target terminal sequence so as to uniquely identify the protein. To this end, the identification possibility predictor refers to the terminal sequences of the extracted protein candidates and determines the kinds of amino-acid residues that can be added to the target terminal sequence. This is because amino-acid residues that can be added to the target terminal sequence should be included in the terminal sequences of the extracted protein candidates.

For example, in the case where a sequence created by adding one certain amino-acid residue to the target terminal sequence corresponds to only one protein among the extracted protein candidates, it can be said that the protein will be uniquely identified by using the target terminal sequence with the additional amino-acid residue. When there are multiple kinds of amino-acid residues that can be added to the target terminal sequence, the identification possibility predictor makes a similar judgment for each type of amino-acid residue. If the protein cannot be uniquely identified by the addition of one amino-acid residue, another amino-acid residue is added to the amino-acid sequence to further extend the sequence. Upon reaching a state in which the protein can be uniquely identified for any of the amino-acid residues that can be added, the identification possibility predictor determines, for example, the maximum number of the added amino-acid residues, i.e. the maximum extension length of the amino-acid sequence. This information is important since it does not merely indicate that the protein cannot be uniquely identified from the currently set target terminal sequence; it also shows how many amino-acid residues need to be additionally determined by an experiment or other methods in order to uniquely determine the protein. The system presents this result to the analysis operator, for example, by showing it on a display unit. Thus, the analysis operator is provided with useful information for protein identification even if the protein cannot be uniquely identified by the given target terminal sequence (e.g. the sequence specified by the analysis operator).

Theoretically, it is possible to add an unlimited number of amino-acid residues until the state in which the protein can be uniquely identified is reached. However, the length of the amino-acid sequence of a peptide that can be predicted from a result of a mass spectrometry is actually limited by the mass accuracy of the mass spectrometry. Even if a conclusion is obtained which suggests that the protein can be uniquely identified by extending the amino-acid sequence to a certain length, the conclusion is practically useless for the analysis operator if the suggested length exceeds the limit that can actually be derived from a result of a mass spectrometry.

Accordingly, in the protein identification method according to the present invention, the identification possibility prediction step may preferably be performed in such a manner that, in the case where the protein cannot be uniquely identified even if the terminal sequence created by adding amino-acid residues to the target terminal sequence is extended to an upper-limit length, it is concluded that the protein cannot be identified from the terminal sequence. This method prevents a waste of time for unnecessary data processing in the case where no practically useful information is expected to be presented to the analysis operator.

The “information in which various known kinds of proteins are linked with, at least, their respective terminal sequences” to be referenced in the identification candidate extraction step may be a protein database in which each protein is linked with its entire amino-acid sequence. However, such a database is enormously large in size and inevitably requires a long period of time in the search for proteins having the target terminal sequence or the extended terminal sequence created by adding amino-acid residues to the target terminal sequence. For the present invention, it is unnecessary to have information about the entire amino-acid sequence of each protein; what is minimally required is a database in which each protein is linked with the amino-acid sequence of its terminal region having a certain length.

Accordingly, in one preferable mode of the protein identification method according to the present invention, a terminal sequence database creation step is further provided, in which, based on amino-acid sequence information of known kinds of proteins, an amino-acid sequence of a preset length of a terminal region of each protein is extracted and a terminal sequence database in which the extracted terminal sequence is linked with the corresponding protein is created, and

the candidate extraction step is performed in such a manner that a given terminal sequence is compared with each of the terminal sequences contained in the terminal sequence database so as to find a protein corresponding to the given terminal sequence and extract the protein as a protein candidate.

By this method, the search for the protein concerned can be quickly performed and the data processing time for protein identification can be shortened. The terminal sequence database may be created at an appropriate point in time before the identification is carried out or at the beginning of the identification process.

### Advantageous Effects of the Invention

With the protein identification method and system according to the present invention, even if a protein having a given terminal sequence (e.g. a terminal sequence revealed as a result of a mass spectrometry) in its terminal region cannot be uniquely determined based on that terminal sequence, it is possible to provide the analysis operator with useful information for identifying the protein, which indicates whether the protein can be uniquely identified by additionally revealing the kinds of a certain number of amino-acid residues or the protein cannot be uniquely identified without using any information other than the terminal sequence. For example, such information can be used by the analysis operator in determining a plan of future experiments for protein identification.

In the case of using the UniProt database or a similar database in which proteomes of representative kinds of organisms are registered, the database may additionally contain information on their splicing variants, i.e. the amino-acid sequences of different proteins produced from the same gene. Using such a database or creating the terminal sequence database from such a database opens up the possibility of performing a task which has been conventionally difficult, i.e. the task of identifying a specific variant of protein, in the case where there are different terminal sequences due to the presence of splicing variants.

### BRIEF DESCRIPTION OF DRAWINGS

FIG. 1 is an overall configuration diagram of a protein identification system as one embodiment of the system for carrying out the protein identification method according to the present invention.

FIG. 2 is a flowchart showing a procedure for identifying a protein in the protein identification system of the present embodiment.

FIG. 3 shows (a) an example of a specified terminal sequence and (b) an example of the protein sequences extracted from a database for the specified terminal sequence.

FIG. 4 illustrates a process of performing a sequence prediction for protein identification based on the terminal sequence shown in FIG. 3(a).

FIG. 5 is an overall configuration diagram of a protein identification system as one variation of the present invention.

FIG. 6 is an overall configuration diagram of a protein identification system as another variation of the present invention.

### DESCRIPTION OF EMBODIMENTS

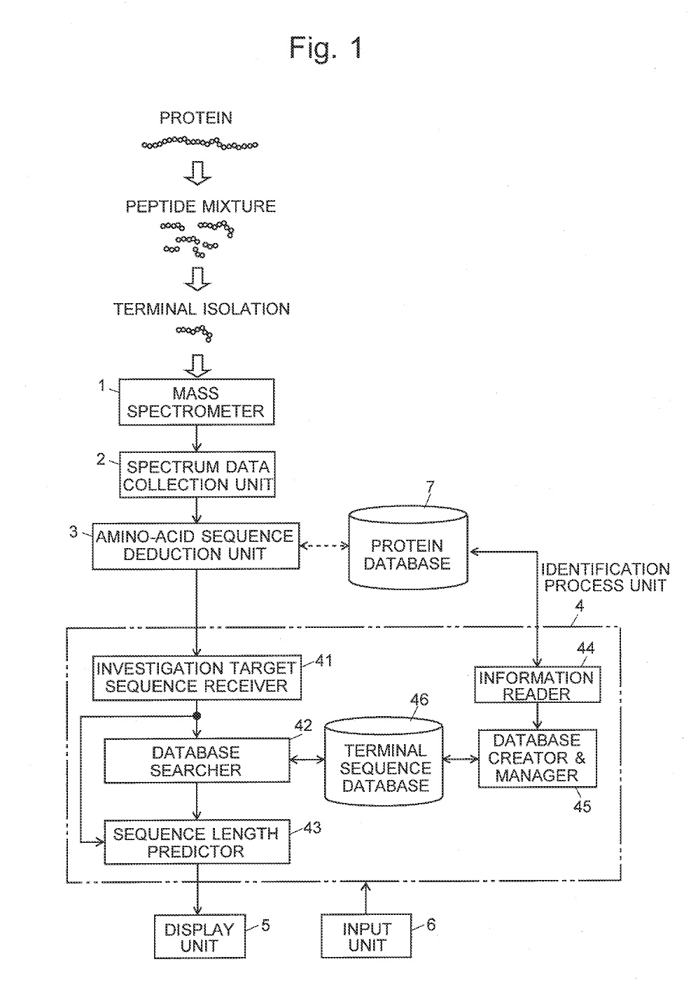

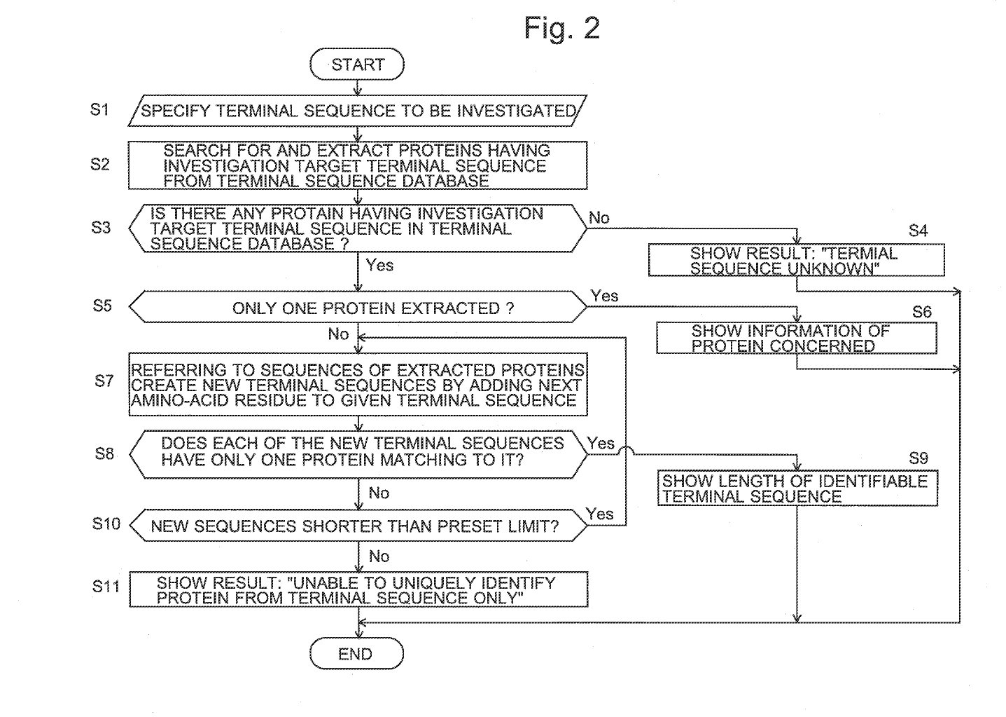

One embodiment of the protein identification system for carrying out the protein identification method according to the present invention is hereinafter described with reference to the attached drawings. FIG. 1 is an overall configuration diagram of the protein identification system of the present embodiment, and FIG. 2 is a flowchart showing a procedure for identifying a protein in the present system.

The protein identification system of the present embodiment includes a mass spectrometer 1, a spectrum data collection unit 2, an amino-acid sequence deduction unit 3, a protein database 7, an identification process unit 4, a display unit 5 and an input unit 6. The identification process unit 4 is a characteristic element for carrying out the protein identification method according to the present invention. It includes an investigation terminal sequence receiver 41, a database searcher 42, a sequence length predictor 43, an information reader 44, a database creator and manager 45, a terminal sequence database 46 and other functional blocks. The units other than the mass spectrometer 1 can be constructed using a computer and other hardware resources, with the main functions being realized by executing a dedicated controlling and processing software program installed on the computer.

Although the mass spectrometer 1 may have any configuration, it should be taken into account that protein identification requires high mass accuracy and high mass-resolving power and that it is normally necessary to perform an MSⁿ analysis including collision induced dissociation (CID). Therefore, for example, an ion-trap time-of-flight mass spectrometer or TOF/TOF mass spectrometer using an electrospray ionization (ESI) source or a MALDI source is typically used.

The protein to be identified is broken into peptide fragments by a pretreatment using an appropriate digestive enzyme, such as trypsin. The obtained peptide mixture contains N and C-terminal peptides of the protein mixed with internal peptides, i.e. the peptides other than the N and C-terminal peptides. Using a commonly known method for analyzing a protein terminal sequence, spectrum data of the N and/or C-terminal peptide are measured with the mass spectrometer 1. The spectrum data collection unit 2 collects the mass spectrum data and MSⁿ spectrum data acquired with the mass spectrometer 1, and temporarily holds those data. Amino-acid sequences of terminal peptides are mainly deduced from MSⁿ spectrum data (typically, MS² spectrum data). The amino-acid sequences can be determined by de novo sequencing or MS/MS ion search. In the case of using the MS/MS ion search, an ion that is likely to be a molecular ion originating from the peptide is set as the precursor ion and an MS² analysis (in some cases, an MSⁿ analysis with n being equal to or greater than three) is performed, and the thereby obtained MSⁿ peak information is used for the deduction of an amino-acid sequence, as will be described later.

Based on the mass spectrum data and the MSⁿ spectrum data obtained in the previously described manner, the amino-acid sequence deduction unit 3 deduces the amino-acid sequence of the N and/or C-terminal peptide of the protein. For example, in the case of using the MS/MS ion search, the peptide sequence is deduced by examining the degree of matching of the obtained MS² spectrum pattern and an MS² spectrum pattern of each peptide calculated from the protein database 7. Various databases open to the public (e.g. the Swiss-Prot database) can be used as the protein database 7. In the case of using a system capable of a sequence analysis by de novo sequencing, an amino-acid sequence is determined manually (i.e. without relying on a database search) from the MS² spectrum.

In the system for carrying out the protein identification method according to the present invention, it is possible to specify both of C-terminal and N-terminal amino-acid sequences of a protein in question, instead of specifying only one of them as described above. Using both C-terminal and N-terminal amino-acid sequences, the success rate of the identification will be improved.

In Step S7 of the protein identification process according to the present embodiment, as will be described later, only one amino-acid residue is added to a terminal sequence. A multiple number of (e.g., two or three) amino-acid residues can be added so as to improve the identification efficiency.

The amino-acid sequence of the terminal region of the protein deduced from the result of a mass spectrometry of a test sample in the previously described manner is sent to the identification process unit 4 as the target to be investigated. The terminal sequence database 46 in the identification process unit 4 is a database in which the amino-acid sequences of the terminal regions of the proteins registered in a protein database or genome sequence database as described earlier are used as the indices, each index being linked with information, entry name, accession number and the other identifiers of the matching protein (i.e. a protein which has the amino-acid sequence corresponding to the index in its terminal region). The information reader 44, which has the function of receiving external data through the Internet or similar communication lines, collects necessary information from the protein database 7 and/or other databases. The database creator and manager 45 organizes the information received through the information reader 44 to build the terminal sequence database 46, and later on, updates this database 46 when necessary.

A procedure for identifying a target protein using the terminal sequence database 46 in the identification process unit 4 is hereinafter described according to FIG. 2.

First, an analysis operator specifies one terminal sequence to be investigated. For example, when an amino-acid sequence of a peptide including a terminal region of a protein is deduced in the amino-acid sequence deduction unit 3, a plurality of candidates are listed together with an index representing the degree of certainty (e.g. a rank or score). The analysis operator selects one of the candidates and enters a command. Upon receiving this command, the investigation terminal sequence receiver 41 sends the specified terminal sequence (which is hereinafter called the “investigation target terminal sequence”) to the database searcher 42 (Step S1).

The database searcher 42 compares the investigation target terminal sequence with the terminal sequences in the terminal sequence database 46 and extracts all the proteins which include the investigation target terminal sequence (Step S2). Needless to say, when the investigation target terminal sequence is estimated to be an N-terminal peptide, the database searcher 42 compares the investigation target terminal sequence with the N-terminal sequences of the proteins in the terminal sequence database 46. Similarly, when the investigation target terminal sequence is estimated to be a C-terminal peptide, the database searcher 42 compares the investigation target terminal sequence with the C-terminal sequences of the proteins in the terminal sequence database 46.

Next, the database searcher 42 determines whether or not a protein having the investigation target terminal sequence is present in the terminal sequence database 46 (Step S3). If it is determined that no protein having the investigation target terminal sequence is present in the terminal sequence database 46, the given investigation target terminal sequence is most likely to be a protein having a new terminal sequence (which cannot be found in the protein database 7, either). In such a case, it is impossible to identify the protein with the system of the present embodiment. Therefore, a message which says that the terminal sequence (or protein) in question is an unknown kind is shown on the display unit 5 (Step S4), and the entire process is completed.

If it is determined that one or more proteins having the investigation target terminal sequence are present in the terminal sequence database 46 (“Yes” in Step S3), the database searcher 42 determines whether or not there is only one protein extracted (Step S5). If the number of extracted protein is one (“Yes” in Step S5), there is only one protein having the investigation target terminal sequence, which means that the protein can be uniquely identified from the specified terminal sequence. Accordingly, the entry name corresponding to the single extracted protein or various other items of information linked with the entry name (the amino-acid sequence information, protein structure and so on) are read from the terminal sequence database 46 and shown on the display unit 5 (Step S6), and the entire process is completed.

By the previously described processes from Step S1 through S6, a database search using a terminal sequence specified by an analysis operator is performed and an identified protein is presented to the analysis operator (when a protein is uniquely identified). Thus, the basic functions of the system are achieved.

In the case where the number of proteins extracted by the database search is two or more (“No” in Step S5), i.e. when the protein cannot be identified from the terminal sequence specified in Step S1, the operation proceeds to Step S7. The processes from Step S7 through the subsequent steps are aimed at predicting what is the length of the terminal sequence of each protein candidate which needs to be investigated to uniquely identify a protein having a terminal region matching the specified terminal sequence if the analysis operator further continues the task of determining the terminal sequence based on the result of the experiment. Although the type of the next amino-acid residue bonded to the inner end of the terminal sequence deduced from the result of the experiment cannot be identified without actually performing a measurement, it is possible to previously test all the possible cases without performing the actual measurement. This test does not need to be performed for all (twenty) kinds of amino acids whose existence are generally known; the test only needs to be performed for the amino acids (amino-acid residues) included in the terminal sequences whose presence has been confirmed in Step S3 using the terminal sequence database 46, since the search target is originally limited to those terminal sequences which are registered in the terminal sequence database 46 created from the known protein database 7.

Accordingly, the sequence length predictor 43 refers to the amino-acid sequences of the two or more proteins extracted in Step S2 and finds the type of amino-acid residue that can be added to the investigation target terminal sequence given in Step S1. Normally, multiple kinds of amino-acid residues will be found. Then, a new terminal sequence is created by adding each single amino-acid residue thus found (Step S7). If the sequence length of the investigation target terminal sequence is N (where N is an integer equal to or greater than one), the sequence length of the investigation target terminal sequence newly created in Step S7 is N+1. When the process of Step S7 is performed after the operation is returned from Step S10 to Step S7 (as will be described later), the terminal sequence to which an amino-acid residue is to be newly added is the sequence which was newly created by the previous execution of Step S7 and now consists of the investigation target terminal sequence with at least one amino-acid residue added to it.

Subsequently, the sequence length predictor 43 determines, for each of the terminal sequences newly created by the addition of one amino-acid residue, whether or not there is only one protein having the new terminal sequence in its terminal region (Step S8). That is to say, this is a process for determining whether each of the new terminal sequences whose sequence length has been increased by the addition of one amino-acid residue to the given terminal sequence in Step S7 has only one uniquely identifiable protein, or there still remains a terminal sequence from which no protein can be uniquely identified. If “Yes” in Step S8, it means that the protein in question can be uniquely identified by adding one amino-acid residue to the given terminal sequence in Step S7. Accordingly, this fact is explained on the display unit 5, together with the length of the terminal sequence to be reached or the number of amino-acid residues to be added (Step S9), and the entire process is completed. Although no result of protein identification for the initially specified investigation target terminal sequence can be obtained at this stage, the analysis operator can obtain information about how many amino-acid residues need to be revealed in addition to the initially specified investigation target terminal sequence in order to uniquely identify the protein.

If “No” in Step S8, i.e. in the case where the protein cannot be uniquely identified even if the length of the terminal sequence is extended by one amino-acid residue, it is determined whether or not the length of the terminal sequences which have been newly created in Step 7 is shorter than a preset upper limit (Step S10). If the sequence length is shorter than the preset upper limit, the operation returns to Step S7. Thus, Steps S7 through S10 are repeated, whereby amino-acid residues are added, one after another, to the original investigation target terminal sequence. This process may be repeated until the sequence length reaches the preset upper limit. During this process, when a situation in which each of the terminal sequences with different amino-acid residues added has only one uniquely identifiable protein is reached, the operation proceeds from Step S8 to Step S9. On the other hand, in the case where there still remains a terminal sequence from which no protein can be uniquely identified even if the terminal sequence is fully extended by adding amino-acid residues until the sequence length reaches the preset upper limit, the determination result in Step S10 will be “No.” In such a case, it is possible to determine that the protein in question cannot be identified even if the sequence length deduction process for the protein determination is further continued. Accordingly, this fact is explained on the display unit 5 (Step S11), and the entire process is completed.

The “preset upper limit” in Step S10 can be predetermined taking into account the capabilities of the mass spectrometer to be used (e.g. the mass accuracy, mass-resolving power and so on) and/or other information. For example, with modern mass spectrometers, due to the limitations of their performances, it is normally impossible to determine, with high reliability, the amino-acid sequence of a peptide which exceeds an amino-acid sequence length of approximately 20. Accordingly, the “preset upper limit” can be set at “20.” In the future, when mass spectrometers with higher performances are developed and the determination of peptide sequences having longer amino-acid sequence lengths is made possible, the “preset upper limit” can be increased to accordingly higher values.

As described thus far, with the protein identification system of the present embodiment, when the protein in question can be uniquely identified from a terminal sequence given as an investigation target based on the result of an experiment or other information, this fact is explained on the display unit 5. Even if the protein cannot be uniquely identified using the investigation target terminal sequence, the result of a prediction about how many amino-acid residues need to be additionally revealed in order to uniquely identify the protein is shown on the display unit 5. In the case where there is no possibility that the protein will be uniquely identified if the terminal sequence is fully extended to the preset upper limit, this fact is explained on the display unit 5. Thus, even if the protein cannot be identified, useful information for future experiments will be provided to the analysis operator.

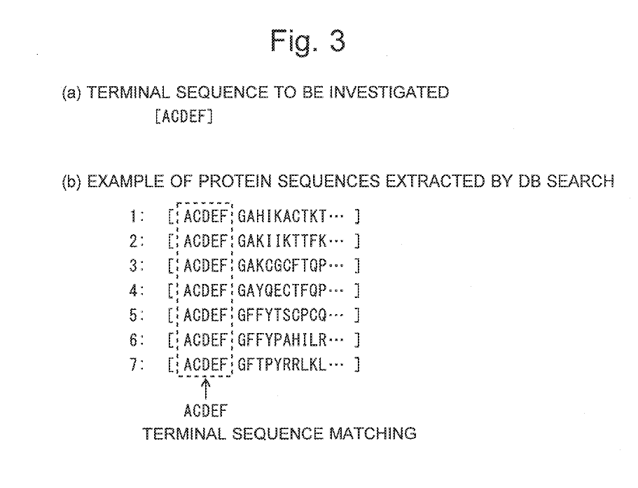

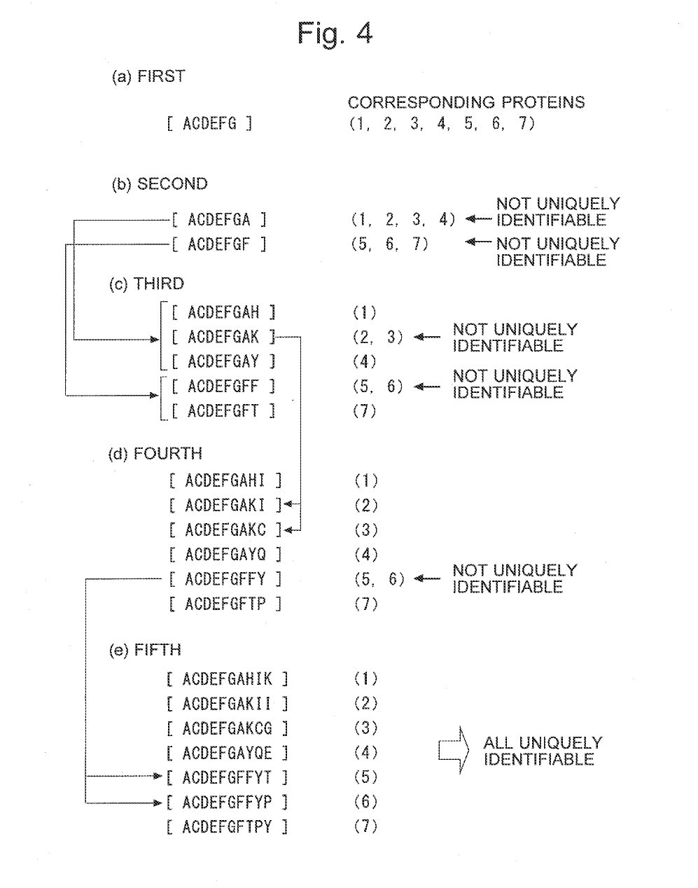

A specific example of the protein identification process shown in FIG. 2 is hereinafter described by means of FIGS. 3 and 4.

As shown in FIG. 3(a), the investigation target terminal sequence in the present example is a peptide which consists of five amino-acid residues [ACDEF] bonded together and is estimated to be an N-terminal peptide. Now, suppose that seven proteins as shown in FIG. 3(b) have been extracted as a result of the database search in Step S2 performed for this investigation target terminal sequence. FIG. 3(b) shows the amino-acid sequence composed of 15 amino-acid residues in the N-terminal region of each of the seven proteins. Naturally, the entire length of each protein is much longer than these terminal regions. It should be noted that those amino-acid sequences are imaginary ones created for convenience of explanation and do not actually exist.

Any of the seven proteins shown in FIG. 3(b) has the investigation target terminal sequence [ACDEF] in its N-terminal region. Therefore, the determination result in Step S3 is “Yes”, while the determination result in Step S4 is “No.” That is to say, this is an example in which the protein cannot be uniquely identified from the investigation target terminal sequence initially specified by the analysis operator.

When Step S7 is performed for the first time immediately after Step S5, the given terminal sequence is [ACDEF], i.e. the investigation target terminal sequence. For this sequence, the sixth amino-acid residue (as counted from the N-terminal) to be bonded next to the innermost amino-acid residue “F” is to be identified. If the sixth amino-acid residue from the N-terminal is theoretically investigated without relying on an experiment, all of the 20 kinds of amino acids can be selected as the candidates. However, it is actually meaningless to investigate amino-acid sequences that are not present in the terminal sequence database 46. Therefore, in the present case, only the terminal sequences which are registered in the terminal sequence database 46 need to be searched for an amino-acid residue to be bonded to the sixth place from the N-terminal. This can eventually be reduced to the question of whether or not the seven proteins extracted in Step S2 can be further narrowed down. That is to say, only the amino-acid residues at the sixth place from the N-terminals in the amino-acid sequences of the seven proteins need to be examined as the candidates of the amino-acid residue to be added at the sixth place from the N-terminal.

As shown in FIG. 3(b), the amino-acid residues at the sixth place from the N-terminals are all “G.” Therefore, when Step S7 is performed for the first time, only one kind of terminal sequence, [ACDEFG], is newly created by adding “G” to [ACDEF] (see FIG. 4(a)). Since this terminal sequence [ACDEFG] is included in any of the seven proteins, the determination result in Step S8 is “No.” Now, suppose that the “present upper limit” in Step S10 is set at “20” as described earlier. The current length of the terminal sequence is “6” and less than “20.” Accordingly, the determination result in Step S10 is “Yes” and the operation returns to Step S7.

When Step S7 is performed for the second time, the given terminal sequence is [ACDEFG]. Based on the amino-acid sequences of the seven proteins shown in FIG. 3(b), it can be found that there are two kinds of amino-acid residues, “A” and “F”, which can be selected as the seventh amino-acid residue (as counted from the N-terminal) to be added next to the innermost amino-acid residue “G” of the given terminal sequence. Therefore, the terminal sequences newly created in Step S7 are the two terminal sequences [ACDEFGA] and [ACDEFGF] which are created by adding “A” and “F” to [ACDEFG], respectively. The former terminal sequence is included in each of the proteins Nos. 1, 2, 3 and 4 shown in FIG. 3(b), while the latter is included in each of the proteins Nos. 5, 6 and 7 (see FIG. 4(b)). At this stage, it is still impossible to uniquely determine the protein, since four or three kinds of proteins remain as the candidates even if either [ACDEFGA] or [ACDEFGF] is experimentally determined as the terminal sequence.

Subsequently, the operation once more returns from Step S10 to Step S7, where the following five new terminal sequences are similarly created: [ACDEFGAH], [ACDEFGAK], [ACDEFGAY], [ACDEFGFF] and [ACDEFGFT] (see FIG. 4(c)). At this stage, for example, if [ACDEFGAH] or [ACDEFGAY] is experimentally obtained as the terminal sequence, the protein can be uniquely identified since there is only one kind of protein in either case. On the other hand, if the experimentally determined terminal sequence is [ACDEFGAK], it is impossible to determine whether this sequence corresponds to the protein No. 2 or 3. Therefore, the determination result in Step S8 is still “No”, and the operation once more returns through Step S10 to Step S7.

Such processes are repeated, and when Step S7 is performed for the fifth time, the following seven terminal sequences are newly created: [ACDEFGAHIK], [ACDEFGAKII], [ACDEFGAKCG], [ACDEFGAYQE], [ACDEFGFFYT], [ACDEFGFFYP], and [ACDEFGFTPY]. This time, since each terminal sequence is linked with one protein (see FIG. 4(e)), the determination result in Step S8 is “Yes.” Therefore, the length of the terminal sequence at this point in time, i.e. 10, is shown on the display unit 5, and the entire process is completed. Instead of the sequence length, a piece of information equivalent to the sequence length may be displayed, such as a message which says that “five more amino-acid residues need to be additionally determined to assuredly identify the protein.”

Thus, even if the protein has not been successfully identified, the analysis operator can obtain useful information for conducting experiments in the future or preparing an experiment plan.

Although the descriptions thus far have only dealt with the case of an N-terminal amino-acid sequence, it is evident that the same method can be used to deal with a C-terminal amino-acid sequence.

[Variation 1]

In the protein identification system of the previous embodiment, the database search using the terminal sequence database 46 is performed after the amino-acid sequence of a terminal region of a protein is determined by a mass spectrometric experiment. This system can be modified so that the protein identification is performed using the values of mass-to-charge ratios m/z of the ions originating from a peptide determined from the result of a mass spectrometry.

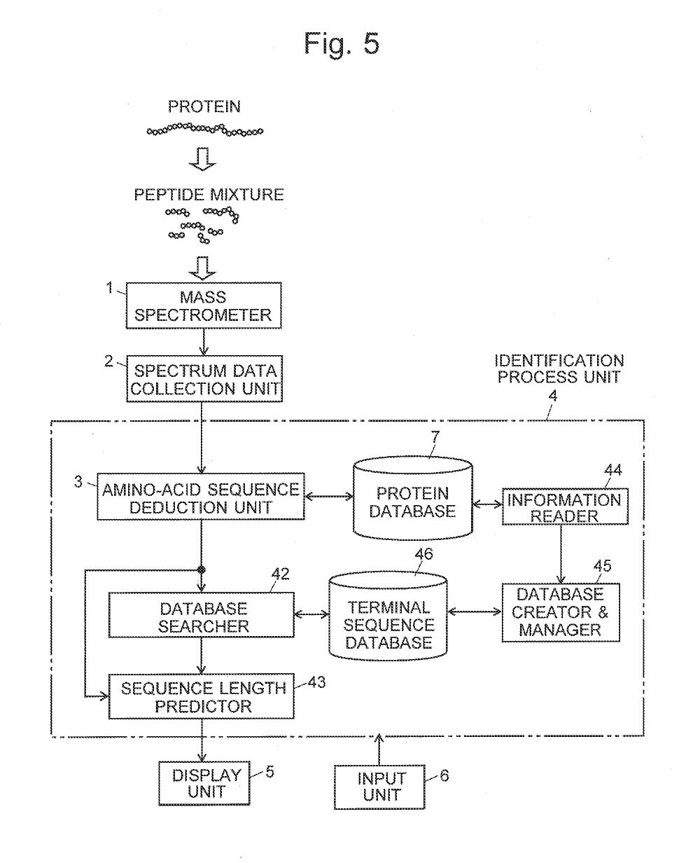

Such a system can be created from the system shown in FIG. 1 by replacing the investigation terminal sequence receiver 41 with a commonly used database search engine and configuring the system so that the protein database 7 can be searched through this database search engine. FIG. 5 shows one example, in which the functions of the amino-acid sequence deduction unit 3 and the protein database 7 are incorporated into the identification process unit 4. Using the result (spectrum peak information) of a peptide mass finger printing or peptide fragment finger printing performed by the spectrum data collection unit 2, each protein having an amino-acid sequence matching the spectrum peak information is extracted from the protein database 7, and furthermore, each terminal sequence in which a peptide corresponding to the spectrum peak information is located in its terminal region is extracted from the terminal sequence database 46. After the processes corresponding to Steps S1 and S2 are thus completed, the processes of Step S3 and subsequent steps can be performed in the previously described way.

[Variation 2]

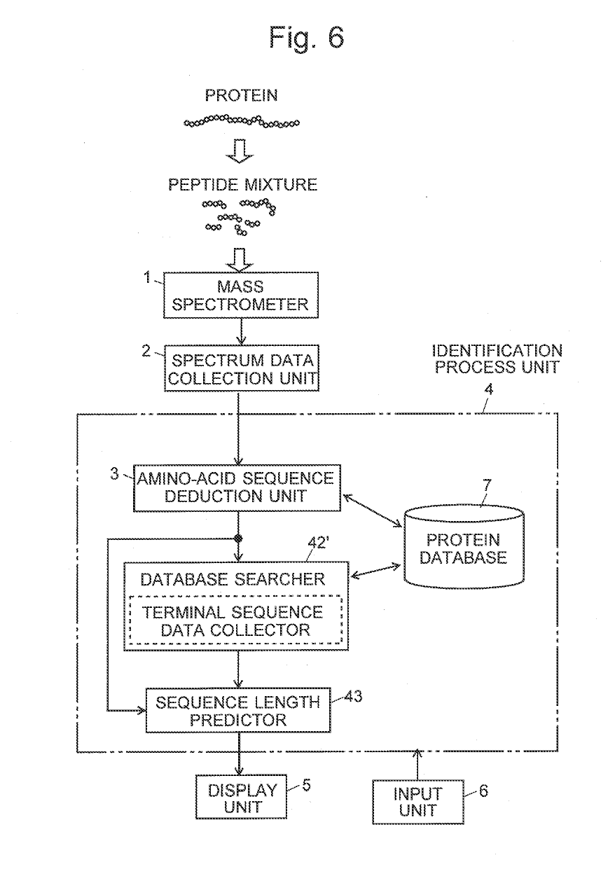

There are an enormous number of proteins whose amino-acid sequences have been entirely known. A protein database containing such information has a massive size that exceeds 10 GB. The data size is expected to further increase in the future. Taking such a situation into account, in the system of the previous embodiment, a minimal set of information necessary for linking terminal sequences with proteins is extracted from the protein database 7 and compiled into a separate database, i.e. the terminal sequence database 46. However, depending on future advancements in hardware technologies, such as the development of storage devices with higher capacities and higher speeds using solid state drives (SSD) or the like, it may become possible to realize a process similar to the system of the previous embodiment by directly searching the protein database 7, in which case the separate terminal sequence database 46 is no longer necessary. FIG. 6 shows one example of such a system configuration.

The present system includes a database searcher 42′ which corresponds to the database searcher 42 into which the function of the database creator and manager 45 is incorporated as the terminal sequence data collector. Both the amino-acid sequence deduction unit 3 and the database searcher 42′ access the protein database 7 to perform their respective processes. For every execution of the database search, the database searcher 42′ performs a process (task) which is performed by the database creator and manager 45 in creating the terminal sequence database 46 in the previous embodiment.

It should be noted that the previously described embodiment and variations are mere examples of the present invention. Any change, modification, addition or the like appropriately made within the scope of the present invention will naturally fall within the scope of claims of the present patent application.

### REFERENCE SIGNS LIST

1 . . . Mass Spectrometer
2 . . . Spectrum Data Collection Unit
3 . . . Amino-Acid Sequence Deduction Unit
4 . . . Identification Process Unit
41 . . . Investigation Target Sequence Receiver
42 . . . Database Searcher
43 . . . Sequence Length Predictor
44 . . . Information Reader
45 . . . Database Creator and Manager
46 . . . Terminal Sequence Database
5 . . . Display Unit
6 . . . Input Unit
7 . . . Protein Database

1. A protein identification method for identifying a protein by a terminal sequence which is an amino-acid sequence of a terminal region of the protein, the method comprising:
a) an identification candidate extraction step, in which a protein corresponding to a target terminal sequence given as a target to be investigated is searched for and extracted as a protein candidate, using information in which various known kinds of proteins are linked with, at least, their respective terminal sequences;
b) an identification result determination step, in which, when only one protein candidate is extracted in the identification candidate extraction step, the candidate is selected as a protein identification result corresponding to the target terminal sequence; and
c) an identification possibility prediction step, in which, when a plurality of protein candidates are extracted in the identification candidate extraction step, an extended length of the terminal sequence with which the protein can be uniquely identified is predicted by determining an amino-acid residue that can be further added to the target terminal sequence with reference to the terminal sequences of the plurality of protein candidates, adding the amino-acid residue to the target terminal sequence to create an extended terminal sequence, and determining, for each extended terminal sequence thus created, whether or not the proteins having the extended terminal sequence can be refined to a single candidate.

2. The protein identification method according to claim 1, wherein:
the identification possibility prediction step is performed in such a manner that, in a case where the protein cannot be uniquely identified even if the terminal sequence created by adding amino-acid residues to the target terminal sequence is extended to an upper-limit length, it is concluded that the protein cannot be identified from the terminal sequence.

3. The protein identification method according to claim 1, wherein:
a terminal sequence database creation step is further provided, in which, based on amino-acid sequence information of known kinds of proteins, an amino-acid sequence of a preset length of a terminal region of each protein is extracted and a terminal sequence database in which the extracted terminal sequences are linked with the corresponding proteins is created; and
the candidate extraction step is performed in such a manner that a given terminal sequence is compared with each of the terminal sequences contained in the terminal sequence database so as to find a protein corresponding to the given terminal sequence and extract the protein as a protein candidate.

4. The protein identification method according to claim 2, wherein:
a terminal sequence database creation step is further provided, in which, based on amino-acid sequence information of known kinds of proteins, an amino-acid sequence of a preset length of a terminal region of each protein is extracted and a terminal sequence database in which the extracted terminal sequences are linked with the corresponding proteins is created; and
the candidate extraction step is performed in such a manner that a given terminal sequence is compared with each of the terminal sequences contained in the terminal sequence database so as to find a protein corresponding to the given terminal sequence and extract the protein as a protein candidate.

5. A protein identification system for identifying a protein by a terminal sequence which is an amino-acid sequence of a terminal region of the protein, the system comprising:
a) an identification candidate extractor for searching a database for a protein corresponding to a target terminal sequence given as a target to be investigated using information in which various known kinds of proteins are linked with, at least, their respective terminal sequences, and for extracting the protein as a protein candidate;
b) an identification result determiner for, when only one protein candidate is extracted by the identification candidate extractor, determining the protein candidate is selected as a protein identification result corresponding to the target terminal sequence; and
c) an identification possibility predictor for, when a plurality of protein candidates are extracted by the identification candidate extractor, predicting an extended length of the terminal sequence with which the protein can be uniquely identified by determining an amino-acid residue that can be further added to the target terminal sequence with reference to the terminal sequences of the plurality of protein candidates, adding the amino-acid residue to the target terminal sequence to create an extended terminal sequence, and determining, for each extended terminal sequence thus created, whether or not the proteins having the extended terminal sequence can be refined to a single candidate.

6. The protein identification system according to claim 5, wherein:
the identification possibility predictor is configured in such a manner that, in a case where the protein cannot be uniquely identified even if the terminal sequence created by adding amino-acid residues to the target terminal sequence is extended to an upper-limit length, it is concluded that the protein cannot be identified by the terminal sequence.

7. The protein identification system according to claim 5, wherein:
a terminal sequence database creator is further provided, by which, based on amino-acid sequence information of known kinds of proteins, an amino-acid sequence of a preset length of a terminal region of each protein is extracted and a terminal sequence database in which the extracted terminal sequences are linked with the corresponding proteins is created; and
the candidate extractor is configured in such a manner that a given terminal sequence is compared with each of the terminal sequences contained in the terminal sequence database so as to find a protein corresponding to the given terminal sequence and extract the protein as a protein candidate.

8. The protein identification system according to claim 6, wherein:
a terminal sequence database creator is further provided, by which, based on amino-acid sequence information of known kinds of proteins, an amino-acid sequence of a preset length of a terminal region of each protein is extracted and a terminal sequence database in which the extracted terminal sequence is linked with the corresponding protein is created; and
the candidate extractor is configured in such a manner that a given terminal sequence is compared with each of the terminal sequences contained in the terminal sequence database so as to find a protein corresponding to the given terminal sequence and extract the protein as a protein candidate.
